(19) United States
(12) Patent Application Publication
(10) Pub. No.: US 2011/0147218 A1
(43) Pub. Date: Jun. 23, 2011

(54) SOLUBLE CADHERIN 17 FOR THE DIAGNOSIS AND RISK STRATIFICATION OF CANCER AND TUMOR OF THE GASTROINTESTINAL TRACT

(76) Inventors: Helmut Meyer, Recklinghausen (DE); Hanna Diehl, Bochum (DE); Susanne Klein-Scory, Wittenberg (DE); Wolff Schmiegel, Bochum (DE); Irmgard Schwarte-Waldhoff, Mettmann (DE); Kai Stühler, Bochum (DE); Jakob Weiss, Bochum (DE)

(21) Appl. No.: 12/670,533

(22) PCT Filed: Jul. 28, 2008
(86) PCT No.: PCT/DE08/01220

§ 371 Date: Mar. 3, 2011

(51) Int. Cl. G01N 33/574; G01N 33/559

(52) U.S. Cl. 204/461; 204/612

(57) ABSTRACT

The invention relates to a method for the diagnosis and risk stratification of a tumor or cancer of the gastrointestinal tract, particularly colon tumor or colon cancer, wherein a determination is made on at least one patient using the novel soluble cadherin 17 biomarker—a proteolytic cleavage product of cadherin 17 having SEQ ID No. 1 or SEQ ID No. 2—or partial peptides and fragments thereof.

The invention relates to a method for the diagnosis and risk stratification of cancer and tumors of the gastrointestinal tract, preferably colon tumor or colon cancer, wherein a determination is carried out on at least one patient using the novel soluble cadherin 17 biomarker—a proteolytic cleavage product of cadherin 17 having SEQ ID No. 1 or SEQ ID No. 2 —or partial peptides and fragments thereof.

Cadherins (calcium-dependent adhesion molecules) are calcium ion-dependent transmembrane glycoproteins from the group of adhesion proteins. They are found predominantly is desmosomes and adherens junctions and mediate cell adhesion in different tissues. Cadherins play a role especially in the stabilization of cell-cell adhesion, embryonal morphogenesis, maintenance of cell polarity and differentiation, and signal transduction.

To date, more than 80 cadherins have been identified in humans. Common to all cadherins are the multiple extracellular cadherin domains (EC). A cadherin domain has approximately 110 amino acids, is evolutionary very conserved, and comprises negatively charged sequence motifs, which mediate calcium ion-dependent, homophilic binding. By short linker sequences approximately ten amino acids long, these ECs are tandemly repeated between 5 and 34 times, wherein the ECs are numbered sequentially starting at the N-terminal end. The superfamily of cadherins falls into six groups according to the number of cadherin domains, the cytoplasmic domain and the size of a cadherin, and gene clusters: classical cadherins, desmosomal cadherins, protocadherins, protein kinase cadherins, FAT-like cadherins, and seven-pass transmembrane (7-TM) cadherins. An individual transmembrane domain, and finally an intracellular C-terminal domain, follow the N-terminal cadherin domains. The classical cadherins, with E-cadherin being the most widely known representative, and the desmosomal cadherins (desmogleins, desmocollins) have five extracellular cadherin repeats and a highly conserved intracellular domain approximately 160 amino acids long. This cytoplasmic domain plays an important role functionally both in cell adhesion and in signal cascades. By way of the interaction with cytoplasmic proteins (catenins or plakoglobin and the like), the cytoplasmic domains of the classical and desmosomal cadherins regulate the close contact with the actin cytoskeleton or with the intermediate filaments.

The expression of cadherin 17 in colorectal carcinomas has been addressed so far in two papers: Hinoi et al. [Hinoi T, Lucas P C, Kuick R, Hanash S, Cho K R, Fearon E R: CDX2 regulates liver intestine-cadherin expression in normal and malignant colon epithelium and intestinal metaplasia. Gastroenterology 2002, 123:1565-1577] report of a continuing high expression of adherin 17 in 24 of 25 carcinomas of the differentiated type. A loss of the cadherin 17 expression in conjunction with the loss of the Cdx2 expression can be found in the colon tumors referred to as LDMDC (large cell minimally differentiated carcinoma) by the work group. In collectively 45 colorectal tumors, Takamura et al. found cadherin 17 expression to be preserved at normal levels in 62% of the tumors [Takamura M, Ichida T, Matsuda Y, Kobayashi M, Yamagiwa S, Genda T, Shioji K, Hashimoto S, Nomoto M, Hatakeyama K, Ajioka Y, Sakamoto M, Hirohashi S, Aoyagi Y: Reduced expression of liver-intestine cadherin is associated with progression and lymph node metastasis of human colorectal carcinoma. Cancer Lett 2004, 212:253-259] and marginally reduced expression in 17 tumors (38%).

Soluble cadherin 17 as a biomarker for cancer and tumors of the gastrointestinal tract, particularly colon tumors, however, has not been described in the prior art.

Furthermore, it is known that soluble E-cadherin has been detected in culture supernatants of human breast cancer cells (Damsky C H, Richa J, Solter D, Knudsen K, Buck C A: Identification and purification of a cell surface glycoprotein mediating intercellular adhesion in embryonic and adult tissue. Cell 1983, 34:455-466; Wheelock M J, Buck C A, Bechtol K B, Damsky C H: Soluble 80-kd fragment of cell-CAM 120/80 disrupts cell-cell adhesion. J Cell Biochem 1987, 34:187-202).

In the years thereafter, assays (primarily EIA and ELISA) were developed for soluble E-cadherin in body fluids, such as blood and urine. Soluble E-cadherin can indeed already be detected in the serum of healthy controls. A significant increase in the concentration of soluble E-cadherin was described for patients with tumors, including colorectal tumors. Since E-cadherin per se is not overexpressed in tumors (at times the expression level is even reduced), this increase in the serum levels of soluble E-cadherin is attributed to the higher proteolytic activity as part of the carcinogenic process. In addition, cellular and histological changes that take place over the course of the carcinogenesis process (dissolution of intercellular junctions and cell-matrix adhesion, dissolution of the basal lamina) could result in a changed transport and metabolism of the ectodomains (Charalabopoulos K, Gogali A, Dalavaga Y, Daskalopoulos G, Vassiliou M, Bablekos G, Karakosta A, Constantopoulos S: The clinical significance of soluble E-cadherin in nonsmall cell lung Cancer. Exp Oncol 2006, 28:83-85; Chan A O, Chu K M, Lam S K, Cheung K L, Law S, Kwok K F, Wong W M, Yuen M F, Wong B C: Early prediction of tumor recurrence after curative resection of gastric carcinoma by measuring soluble E-cadherin. Cancer 2005, 104:740-746; Wilmanns C, Grossmann J, Steinhauer S, Manthey G, Weinhold B, Schmitt-Graff A, von Specht B U: Soluble serum E-cadherin as a marker of tumour progression in colorectal cancer patients. Clin Exp Metastasis 2004, 21:75-78; Gofuku J, Shiozaki H, Doki Y, Inoue M, Hirao M, Fukuchi N, Monden M: Characterization of soluble E-cadherin as a disease marker in gastric cancer patients. Br J Cancer 1998, 78:1095-1101, Katayama M, Hirai S, Kamihagi K, Nakagawa K, Yasumoto M, Kato I: Soluble E-cadherin fragments increased in circulation of cancer patients. Br J Cancer 1994, 69:580-585).

The disadvantage of E-cadherin, however, is that this marker is not tissue-specific. Instead, E-cadherin is expressed in numerous epithelial cells of different organs; soluble E-cadherin is even present already in the serum of healthy controls in a considerable concentration and is measured in increased concentrations in the serum for different tumor entities.

However, there is a tremendous need to provide biomarkers which exhibit high sensitivity and specificity as well as organ-specific selectivity, thereby enabling a reliable diagnosis.

An object of the invention is therefore to develop a method for the diagnosis and risk stratification of a tumor or cancer of the gastrointestinal tract, particularly colon tumor or colon cancer, which enables an improved early diagnosis.

The object is achieved by a method for the diagnosis and risk stratification of a tumor or cancer of the gastrointestinal tract, particularly colon tumor or colon cancer, wherein a determination is made using the novel scadherin 17 biomarker, specifically soluble cadherin 17 (proteolytic cleavage product) having SEQ ID No. 1 or SEQ ID No. 2, or partial peptides and fragments thereof (hereinafter referred to as “scadherin 17” or “soluble cadherin 17”).

According to the invention, the diagnosis and risk stratification of a colon tumor or colon cancer are preferred, wherein a determination is made using the claimed scadherin 17 biomarker (proteolytic cleavage product) having SEQ ID No. 1 or SEQ ID No. 2, or partial peptides and fragments thereof, because high selectivity and significance can be achieved.

In contrast to E-cadherin, scadherin 17 is characterized by extraordinarily high tissue specificity for the colon epithelium. Furthermore, due to increased and/or modified proteolytic activity, tumors exhibit modified “ectodomain shedding” of scadherin 17 with respect to normal colon epithelial cells. As a result, scadherin 17 is a suitable specific biomarker and preferably selective for a colon tumor or colon cancer.

In addition, contrary to the other cadherins, cadherin 17 has seven cadherin repeats, but only a very short characteristic cytoplasmic domain of 21 amino acids; as a result, the difference in size between the transmembrane protein and the soluble form is low compared to E-cadherin. Interacting cytoplasmic proteins are not known; nonetheless, scadherin 17 is apparently functionally active in intercellular adhesion solely through the interaction of the extracellular domains. Overall, the sequence homology of cadherin 17 with other cadherins is only 20-30%.

Subcellular localization of cadherin 17 differs from other cadherins, which are concentrated at highly structured cell junctions (junctional complexes). Cadherin 17, however, is distributed over the lateral surface of the cells and not associated with the cytoskeleton, so that cadherin 17 exhibits advantageous high lateral mobility inside the cytoplasmic membrane.

Within the context of the present invention, “scadherin 17” shall be understood as a cleavage product according to SEQ ID No. 1 or SEQ ID No. 2 and/or partial peptides or fragments thereof from a sequence of cadherin 17 (SEQ ID No. 3 or SEQ ID No. 4). According to the invention, this comprises the cleavage product (SEQ ID No. 1 or SEQ ID No. 2), and partial peptides or fragments thereof, which is to say “soluble cadherin 17”. This cleavage of the extracellular domain (=cleavage products) of a transmembrane protein is referred to as “ectodomain shedding”. The remaining transmembrane peptide comprises amino acids 786 to 808 of SEQ ID No. 3 or SEQ ID No. 4. As a result, the invention also relates to such amino acid sequences (polypeptides, proteins) the sequence identity or homology of which is 70% and more, preferably 80% and more, particularly preferred 90-95% Q and more with SEQ ID No. 1 or SEQ ID No. 2, or the fragments or partial peptides thereof. Also included are such analogous amino acid sequences which, due to the replacement of one or more amino acids in these sequences, still ensure the desired function of the biomarker according to the invention for the diagnosis of a tumor or cancer of the gastrointestinal tract, particularly a colon tumor or colon cancer (hereinafter collectively referred to as “biomarkers according to the invention”). In a preferred embodiment of the invention, the biomarker according to the invention can be detected (in vitro) in the cell supernatant.

The term “tumor or cancer of the gastrointestinal tract” comprises a tumor or cancer of the organs in the gastrointestinal tract, to include the bile duct, gallbladder, pancreas, stomach and intestine, particularly the small intestine, large intestine and rectum.

Also included are types such as carcinoma or adenoma, furthermore malignant or benign tumors, in particular colorectal tumors at various stages in the entire intestinal tract.

The biomarker according to the invention was identified using differential proteome analysis. For this purpose, proteins were isolated from conditioned media of a) well-differentiated intestinal epithelial cells (adenoma) and b) undifferentiated carcinoma cells and concentrated. The proteins were labeled with a dye and subjected to 2D gel electrophoresis, with isoelectric focusing in the first and SDS gel electrophoresis in the second dimension.

The differential representation (differentiated early tumor cell/undifferentiated carcinoma cells) is provided in the examples and shows the specific accumulation of scadherin 17 in the secretome of tumor cells.

The further evaluation was carried out using LC-ESI-MS (/MS) (liquid chromatography electrospray ionization mass spectrometry). For this purpose, the proteins were first broken down into individual peptide fragments by way of trypsin in the gel in which the samples were previously separated. These fragments were separated from each other using reversed phase HPLC and subjected to mass spectrometry analysis in order to identify the individual peptides. Of course other suitable mass spectrometry methods can be applied as well, such as MALDI-TOF MS.

The invention likewise relates to the identification and stratification of patients who are at increased risk and/or have an unfavorable diagnosis for a tumor or cancer of the gastrointestinal tract, particularly colon tumor or colon cancer, particularly in symptomatic and/or asymptomatic patients.

In a preferred embodiment of the invention, the detection of the scadherin 17 biomarker according to the invention may be followed by further analyses, such as a colonoscopy or imaging methods, as described below, thereby providing early detection of a colon tumor and/or colon cancer.

The method according to the invention thus enables clinical decisions that result in fast success of the therapy and the prevention of fatalities, such as a surgical procedure or the endoscopic resection of the diseased tissue. Such clinical decisions likewise include advanced treatment with drugs for the treatment or therapy of a tumor or cancer of the gastrointestinal tract, particularly colon tumor or colon cancer, wherein a determination of scadherin 17 (such as SEQ ID No. 1 or SEQ ID No. 2), or fragments and partial peptides thereof, is made in a patient to be examined.

The invention therefore likewise relates to a method for the diagnosis of patients having a tumor or cancer of the gastrointestinal tract, particularly colon tumor or colon cancer, for the execution of clinical decisions, such as advanced treatment and therapy using drugs.

A further preferred embodiment of the method according to the invention therefore relates to the diagnosis for prognosis, for early detection and detection by differential diagnosis, for assessment of the severity, and for assessment of the course of the tumor or cancer of the gastrointestinal tract, particularly colon tumor or colon cancer, concomitant with therapy.

In a further preferred embodiment, the invention relates to a method for diagnostics for the early or differential diagnosis or prognosis of a tumor or cancer of the gastrointestinal tract, particularly colon tumor or colon cancer, wherein a determination of the biomarker according to the invention is carried out in a patient to be examined.

In one embodiment of the method according to the invention, tissue samples or body fluids (blood, plasma, secretion, urine) are collected from the patient to be examined, and the diagnosis is made in vitro/ex vivo, which is to say outside of the human or animal body. Based on the determination of the biomarkers according to the invention, high significance for the cancer or tumor of the gastrointestinal tract, particularly colon tumor or colon cancer, is achieved and the diagnosis can be made based on the present quantity or the change thereof (leveling: increase/reduction) in at least one patient sample.

In a further embodiment of the invention, the method according to the invention can be carried out as part of an in-vitro diagnosis using parallel or simultaneous determinations of the biomarker according to the invention (for example, multititer plates having 96 and more wells), wherein the determinations are carried out using at least one patient sample.

In a further embodiment of the invention, the method according to the invention can be carried using 2D electrophoresis, wherein in the first dimension isoelectric focusing and in the second dimension gel electrophoresis are conducted (in the broadest sense, proteomics should be employed for this purpose).

In a further embodiment, the method according to the invention and the determinations thereof can be carried out using a rapid test (such as lateral flow test), be it in an individual or multiple parameter determination.

In a further embodiment, the method according to the invention can be carried out in-vivo, wherein the biomarkers according to the invention, particularly SEQ ID No. 1, or the partial peptides and fragments thereof, in particular of the above cleavage products, particularly soluble cadherin 17, are labeled with a probe, in particular an antibody having a contrast medium and detected using a detector suitable for imaging (“molecular imaging”) (Ralph Weissleder, Molecular Imaging in Cancer, Science, Vol. 312, 1168 (2006)).

The invention further relates to the use of the biomarker according to the invention for the diagnosis and/or prognosis and/or early or differential diagnosis of a tumor or cancer of the gastrointestinal tract, particularly a colon tumor or colon cancer.

In a further preferred embodiment, imaging methods are employed, such as sonography, endosonography, contrast x-rays, angiography, ultrasonic diagnostics, tomography (CT/MRCP/MRI), nuclear magnetic resonance imaging, scintigraphy, subtraction angiography, and endoscopy.

In a further preferred embodiment, an analysis for another cancer and tumor of the gastrointestinal tract by way of the biomarkers according to the invention can be carried out, for example using advanced analysis methods as mentioned above, if there is no indication of a colon tumor or colon cancer.

Another object is to provide a corresponding diagnostic device for carrying out the method according to the invention.

Within the context of the present invention, such a diagnostic device shall be interpreted in particular as an array or assay (such as an immunoassay, ELISA and the like), in particular a protein chip (U.S. Pat. No. 6,346,413 B1, US 2005/0014292), in the broadest sense a device for carrying out the method according to the invention.

The invention further relates to a kit for carrying out the method according to the invention, comprising in particular detection reagents and further media. Such detection reagents comprise antibodies and the like, for example.

The detection and quantification of the biomarker according to the invention can likewise be carried out using further protein diagnostic methods commonly known to the person skilled in the art, particularly with the use of radioactive or fluorescence-labeled antibodies. At this point, in particular suited bioanalytical methods should be mentioned, such as western blotting (1D and 2D), immunohistochemistry, antibody arrays, Luminex, ELISA, immunofluorescence, radio immuno assays and further suitable bioanalytical methods, such as mass spectrometry methods, for example MRM (multiple reaction monitoring) or AQUA (absolute quantification), which can be used to quantitatively measure the biomarkers.

The following examples and figures are provided for a more detailed description of the invention, however without limiting the invention to these examples and figures.

### EXAMPLES AND FIGURES

For the identification of biomarker candidates, the secretome of cultured tumor cells was used. According to a broader definition, the secretome comprises all proteins which are released by the cells into the culture medium by way of different mechanisms.

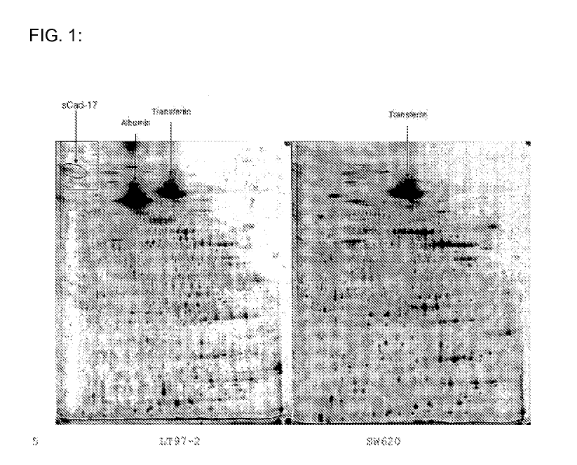

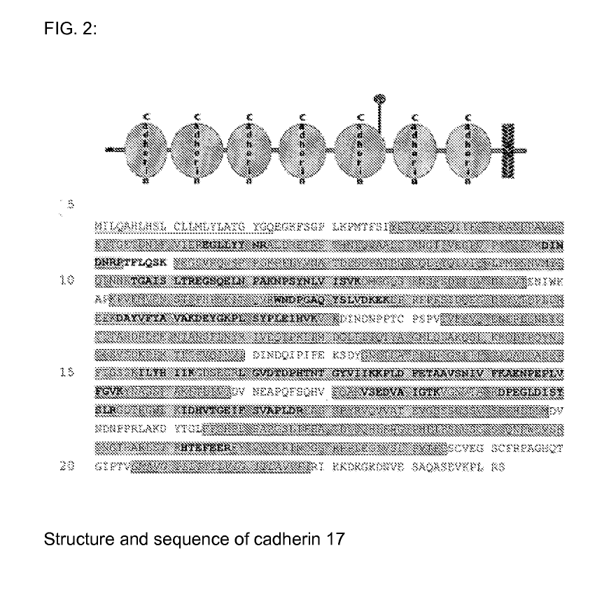

For this purpose, secretomes of human SW620 colon cancer cells (ATCC) and of human LT97-2 intestinal adenoma cells (provided by Mrs. Prof. B. Marian, Vienna) were produced as described (Volmer M W, Radacz Y, Hahn S A, Klein-Scory S, Stuhler K, Zapatka M, Schmiegel W, Meyer H E, Schwarte-Waldhoff I: Tumor suppressor Smad4 mediates downregulation of the antiadhesive invasion-promoting matricellular protein SPARC: Landscaping activity of Smad4 as revealed by a “secretome” analysis. Proteomics 2004, 4:1324-1334; Volmer M W, Stuhler K, Zapatka M, Schoneck A, Klein-Scory S, Schmiegel W, Meyer H E, Schwarte-Waldhoff I: Differential proteome analysis of conditioned media to detect Smad4 regulated secreted biomarkers in colon cancer. Proteomics 2005, 5:2587-2601 and Diehl H C, Stühler K, Klein-Scory S, Volmer M W, Schöneck A, Bieling C, Schmiegel W, Meyer H, Schwarte-Waldhoff I: A catalogue of proteins released by colorectal cancer cells in vitro as an alternative source for biomarker discovery. Proteomics Clin. Appl. 2007, 1:47-61). The secretomes were labeled with CyDye fluorescent dyes, separated on a two-dimensional gel, and the protein samples were read with the laser scanner and represented (FIG. 1). Prominent spots of the LT97-2 secretome were cut out of the gel, tryptically digested, and identified as described by way of mass spectrometry (Diehl et al. (supra)). The chain of protein spots labeled in FIG. 1 was identified as cadherin 17. FIG. 2 shows the protein sequence of cadherin 17, the cadherin domains being highlighted in gray (see diagram); the tryptic peptides that were identified are marked in bold.

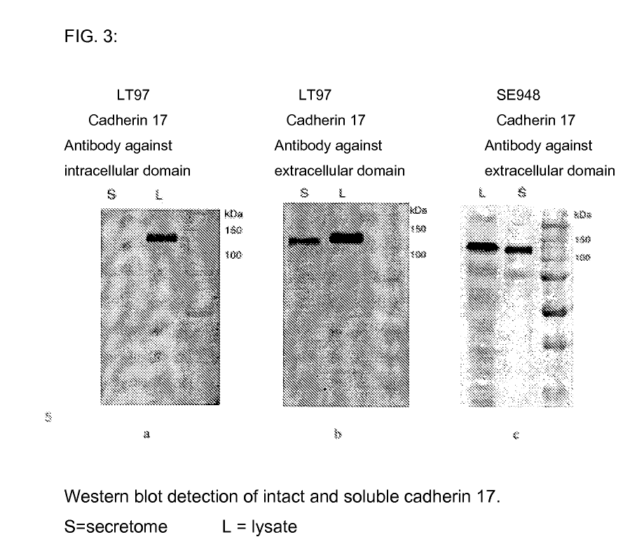

Cadherin 17, like E-cadherin, is a transmembrane protein. Soluble E-cadherin occurs in the secretome through ectodomain shedding, which is to say by the cleavage and release of the extracellular domains. Using western blot analyses of secretomes and lysates of LT97-2 cells, it is shown in FIG. 3 that soluble cadherin 17 is likewise formed by ectodomain shedding and in this way enters the secretome: First, an antibody was used which is directed against the C-terminal intracellular domain of cadherin-17 (sc-6978, Santa Cruz, affinity-purified polyclonal antibody, goat). This antibody detects a protein of the anticipated size only in the cell lysate (FIG. 3a). Furthermore, an antibody was used which is directed against the extracellular domain of cadherin 17 (141713, R&D, monoclonal antibody from the mouse). This antibody in turn detects the same intact transmembrane protein in the cell lysate. In the secretome, this antibody detects a protein that is marginally (approximately 10 kDa) smaller: soluble cadherin 17. A further antibody against the extracellular domain of cadherin 17 (15-288-22817, GenWay, affinity-purified polyclonal antibody, chicken) confirmed this result (FIG. 3b). Other human colon tumor cells also show the soluble cadherin 17 in the secretome (FIG. 3c).

### EXPLANATION OF THE FIGURES

FIG. 1:

On the left, the secretome of LT97-2 cells is shown, on the right the secretome of SW620 cells (separated in the same gel, read separately into the laser scanner). In the LT97-2 secretome, the spot chain is marked which was identified as cadherin 17.

Also marked are transferrin and albumin, which in fact are not secretome components, but added media or residual contamination with albumin from the full medium.

FIG. 2:

Structure and sequence of cadherin 17

The signal peptide is highlighted in dark in the schematic illustration on the left and underlined in the sequence. The seven cadherin domains are highlighted in gray. The transmembrane domain, which in the schematic is filled in a “zipper pattern”, is written in italics in the sequence. The peptides identified from the protein spots shown in FIG. 1 are printed in bold.

FIG. 3:

Western blot detection of intact and soluble cadherin 17.

1. A method for the diagnosis and risk stratification of a tumor or cancer of the gastrointestinal tract, particularly colon tumor or colon cancer, wherein a determination of soluble cadherin 17 according to SEQ ID No. 1 or SEQ ID No. 2 and/or fragments and partial peptides thereof is made on a patient to be examined.

2. The method for the diagnosis and risk stratification of a tumor or cancer of the gastrointestinal tract, particularly colon tumor or colon cancer, according to claim 1, characterized in that the diagnosis is an in-vitro diagnosis.

3. A method for the diagnosis and risk stratification according to claims 1 to 2 for the execution of clinical decisions, particularly advanced treatment and therapy using drugs.

4. A method for the diagnosis and risk stratification according to any one of claims 1 to 3, characterized in that the diagnosis is made for prognosis, early detection and detection by differential diagnosis, assessment of the severity, and assessment of the course of the disease concomitant with the therapy.

5. A method according to any one of the preceding claims, characterized in that parallel or simultaneous determinations of soluble cadherin 17, or fragments and partial peptides thereof, are carried out.

6. A method according to any one of the preceding claims, characterized in that 2D electrophoresis is conducted, wherein isoelectric focusing is carried out in the first dimension and gel electrophoresis in the second dimension.

7. A method according to any one of the preceding claims, characterized in that the determinations are carried out using at least one patient sample.

8. A method according to any one of the preceding claims, characterized in that the determinations are carried out using a rapid test, particularly in individual or multiple parameter determinations.

9. A kit for a diagnosis according to any one of claims 1-8, comprising detection reagents and further media.

10. A diagnostic device for carrying out a method according to any one of claims 1 to 8, particularly a protein biochip, array, or assay.


SEQUENCE LISTING (4 sequences)

SEQ ID NO: 1 - protein, 762 aa, Homo sapiens
EGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTV

SEQ ID NO: 2 - protein, 762 aa, Homo sapiens
EGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTDFEEREYVVLIRINDGGRPPLEGIVSLPVTFCSCVEGSCFRPAGHQTGIPTV

SEQ ID NO: 3 - protein, 832 aa, Homo sapiens
MILQAHLHSLCLLMLYLATGYGQEGKFSGPLKPMTFSIYEGQEPSQIIFQFKANPPAVTFELTGETDNIFVIEREGLLYYNRALDRETRSTHNLQVAALDANGIIVEGPVPITIEVKDINDNRPTFLQSKYEGSVRQNSRPGKPFLYVNATDLDDPATPNGQLYYQIVIQLPMINNVMYFQINNKTGAISLTREGSQELNPAKNPSYNLVISVKDMGGQSENSFSDTTSVDIIVTENIWKAPKPVEMVENSTDPHPIKITQVRWNDPGAQYSLVDKEKLPRFPFSIDQEGDIYVTQPLDREEKDAYVFYAVAKDEYGKPLSYPLEIHVKVKDINDNPPTCPSPVTVFEVQENERLGNSIGTLTAHDRDEENTANSFLNYRIVEQTPKLPMDGLFLIQTYAGMLQLAKQSLKKQDTPQYNLTIEVSDKDFKTLCFVQINVIDINDQIPIFEKSDYGNLTLAEDTNIGSTILTIQATDADEPFTGSSKILYHIIKGDSEGRLGVDTDPHTNTGYVIIKKPLDFETAAVSNIVFKAENPEPLVFGVKYNASSFAKFTLIVTDVNEAPQFSQHVFQAKVSEDVAIGTKVGNVTAKDPEGLDISYSLRGDTRGWLKIDHVTGEIFSVAPLDREAGSPYRVQVVATEVGGSSLSSVSEFHLILMDVNDNPPRLAKDYTGLFFCHPLSAPGSLIFEATDDDQHLFRGPHFTFSLGSGSLQNDWEVSKINGTHARLSTRHTEFEEREYVVLIRINDGGRPPLEGIVSLPVTFCSCVEGSCFRPAGHQTGIPTVGMAVGILLTTLLVIGIILAVVFIRIKKDKGKDNVESAQASEVKPLRS

SEQ ID NO: 4 - protein, 832 aa, Homo sapiens [sequence omitted]